(19) United States
(12) Patent Application Publication
(10) Pub. No.: US 2002/0142942 A1
(43) Pub. Date: Oct. 3, 2002

(54) Method for treating premenstrual or late luteal phase syndrome

(75) Inventors: Bennett M. Shapiro, Pipersville, PA (US); Nadia Rupniak, Bishops Stortford (GB)

Correspondence Address: MERCK AND CO INC, P O BOX 2000, RAHWAY, NJ, 070650907

(73) Assignee: Merck & Co. Inc.

(21) Appl. No.: 10/105,529

(22) Filed: Mar. 25, 2002

Related U.S. Application Data
(63) Continuation of application No. 09/140,042, filed on Aug. 26, 1998.
(60) Provisional application No. 60/057,744, filed on Aug. 28, 1997.

(51) Int. Cl. A61K 31/00

(52) U.S. Cl. 514/1

(57) ABSTRACT

A tachykinin receptor antagonist is useful for alleviating or managing symptoms associated with premenstrual syndrome in a woman, in particular, for the treatment or prevention of disturbances of appetite, disturbances of mood, or both, associated with premenstrual syndrome.

### BACKGROUND OF THE INVENTION

Each month, for a few days prior to the onset of menstruation, many otherwise-healthy women develop symptoms of disturbed mood and appetite known as Premenstrual Syndrome (PMS). This syndrome was first termed “premenstrual tension” by R. T. Frank in 1931 and is generally characterized by nervousness, irritability, emotional instability, depression, and possibly headaches, edema, and mastalgia. PMS occurs during the 7 to 10 days before menstruation and generally disappears a few hours after onset of menstrual flow. PMS is also now referred to as Late Luteal Phase Syndrome (LLS).

Many females report a variety of physical and emotional changes associated with specific phases of the menstrual cycle. For most of these females, these changes are of short duration, cause little distress, and have no effect on social or occupational functioning. Other women have one or more of a broad range of symptoms that temporarily disturb normal functioning. Symptoms last from a few hours to 10 to 12 or more days and usually cease with onset of menses; however, in perimenopausal women, symptoms may persist through and after menses. Type and intensity of symptoms vary in the general population and may also vary in individuals. An essential feature of PMS is a pattern of clinically significant emotional and behavioral symptoms that occur during the last week of the luteal phase and remit within a few days after the onset of the follicular phase. In most females, these symptoms occur in the week before and remit within a few days after the onset of menses.

With onset of menses, PMS is replaced by dysmenorrhea in many women. Significant dysmenorrhea is more common in the teens and tends to diminish with maturity. Conversely, PMS may begin in the 20s and increase with age.

The most common adverse symptoms of women suffering from PMS are mood alteration and psychologic effects: irritability, nervousness, lack of control, agitation, anger, insomnia, difficulty in concentrating, lethargy, depression, anxiety, and severe fatigue. Symptoms related to fluid retention are edema, transient weight gain, oliguria, and breast fullness and pain. Neurologic and vascular symptoms include headache, vertigo, syncope, paresthesias of extremities, easy bruising, and cardiac palpitation. GI symptoms include bloating, constipation, nausea, vomiting, and changes in appetite. Pelvic heaviness or pressure and backache may occur. Skin problems of acne, neurodermatitis, and aggravation of other skin disorders may also occur. Respiratory problems (allergies and infection) and eye complaints (visual disturbance and conjunctivitis) may be worse premenstrually.

Among the most commonly experienced adverse symptoms are marked affective lability (e.g., sudden episodes of tearfulness, sadness, or irritability); persistent feelings of irritability, anger or tension (feeling “on edge”); and feelings of depression, anxiety and self-deprecating thoughts. Also common are decreased interest in usual activities, fatigability and loss of energy, a subjective sense of difficulty in concentrating, changes in appetite, cravings for specific foods (especially carbohydrates), and sleep disturbance. Other physical symptoms, such as breast tenderness or swelling, headaches, joint or muscle pain, a sensation of “bloating,” and weight gain, may also be present.

Premenstrual syndrome seems to be related to fluctuations in estrogen and progesterone. Estrogen exerts fluid-retaining action; transitory increases in fluid in different body tissues seem to explain symptoms such as weight gain, edema, breast tenderness, and possibly, bloating. However, many symptoms do not correlate in intensity with fluid retention and weight gain; eg, diuretics promote sodium and water excretion but do not relieve all of the symptoms and may have no effect on the symptom complex. Estrogen-progesterone imbalance, excessive aldosterone or ADH, carbohydrate metabolism changes, hypoglycemia, hyperprolactinemia, allergy to progesterone, retention of sodium and water by the kidneys, and psychogenic factors have all been implicated.

The neuropeptide receptors for substance P (neurokinin-1; NK-1) are widely distributed throughout the mammalian nervous system (especially brain and spinal ganglia), the circulatory system and peripheral tissues (especially the duodenum and jejunum) and are involved in regulating a number of diverse biological processes. This includes sensory perception of olfaction, vision, audition and pain, movement control, gastric motility, vasodilation, salivation, and micturition (B. Pernow, Pharmacol. Rev., 1983, 35, 85-141). The NK-1 and NK-2 receptor subtypes are implicated in synaptic transmission (Laneuville et al., Life Sci., 42, 1295-1305 (1988)).

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to SP the known mammalian tachykinins include neurokinin A and neurokinin B. The current nomenclature designates the receptors for substance P, neurokinin A, and neurokinin B as neurokinin-1, neurokinin-2, and neurokinin-3, respectively.

Substance P is a pharmacologically-active neuropeptide that is produced in mammals and acts as a vasodilator, a depressant, stimulates salivation and produces increased capillary permeability. It is also capable of producing both analgesia and hyperalgesia in animals, depending on dose and pain responsiveness of the animal (see R. C. A. Frederickson et al., Science, 199, 1359 (1978); P. Oehme et al., Science, 208, 305 (1980)) and plays a role in sensory transmission and pain perception (T. M. Jessell, Advan. Biochem. Psychopharmacol. 28, 189 (1981)).

Known treatment of premenstrual syndrome involves symptomatic relief and, when possible, correcting the cause. Fluid retention may be relieved by reducing sodium intake and using a diuretic (eg, hydrochlorothiazide 50 to 100 mg/day orally), starting just before the time symptoms are usually noted. Non-steroidal anti-inflammatory drugs are administered patients suffering from PMS, but these are only effective for some of the physical symptoms. Counseling about the symptoms can increase understanding and lead to modification of activities for stress reduction. Partner involvement, directly or indirectly, may help both to cope with the PMS. Hormonal manipulation is effective for some women. Possible regimens include (1) oral contraceptives, (2) natural progesterone by vaginal suppository (200 to 400 mg/day) or injection (progesterone in oil 5 to 10 mg IM) for 10 to 12 days premenstrually, or (3) long-acting progestin (eg, medroxyprogesterone acetate 200 mg IM every 2 to 3 mo) to eliminate cyclic changes. Tranquilizers (eg, alprazolam 0.25 mg/day orally at bedtime) are used in patients with irritability, nervousness, and lack of control, especially if they are unable to change their stressful environments. Dietary changes, increasing protein and decreasing sugars as well as supplementing with vitamin B complex (especially pyridoxine and/or magnesium) or employing carbohydrate blends, may be helpful for some women. Preliminary studies have suggested that regimens, using spironolactone (Aldactone, Searle), bromocriptine, or monoamine oxidase (MAO) inhibitors may also be effective in relieving depression and crying spells. Other drugs, including progesterone, lithium carbonate, thiazide, diuretics, antidepressants and bromocyptone (Parlodel, Sandoz), have been tried with uncertain success. More recently, selective serotonin reuptake inhibitors, such as fluoxetine, paroxetine, fluvoxamine, fenfluramine, and combinations thereof, have been examined for the treatment of PMS (see e.g. U.S. Pat. Nos. 4,971,998, 5,114,976, 5,223,540). These approaches have had limited success, however, and a means of treating the mood and appetite disturbances commonly experienced on a recurring basis by a large number of women would be of great benefit.

Neurokinin-1 (NK-1; substance P) receptor antagonists are being developed for the treatment of a number of physiological disorders associated with an excess or imbalance of tachykinins, and in particular substance P. Examples of conditions in which substance P has been implicated include disorders of the central nervous system such as anxiety, depression and psychosis (see, for instance, PCT Patent Publication Nos. WO 95/16679, WO 95/18124 and WO 95/23798). More recently, PCT Patent Publication No. WO 96/24353 suggests that a more efficacious and safe treatment of psychiatric disorders would be achieved using a combination of a tachykinin antagonist and a serotonin agonist or selective serotonin reuptake inhibitor (SSRI). However, such as regimen would not be free of side-effects due to the serotonin agonist or SSRI.

Currently there are only limited means for treating the mood and appetite disturbances commonly experienced on a recurring basis by a large number of women suffering from premenstrual syndrome. In view of the short-comings of existing agents, there is a need for new effective method for alleviating or managing symptoms associated with premenstrual syndrome.

### SUMMARY OF THE INVENTION

The present invention relates to the use of a tachykinin receptor antagonist, in particular a neurokinin-1 receptor antagonist, for the treatment, prevention or amelioration of premenstrual syndrome in a woman comprising the administration of a tachykinin antagonist, in particular a neurokinin-1 receptor antagonist. The present invention is further directed to a method for alleviating or managing symptoms associated with premenstrual syndrome in a woman comprising the administration of a tachylkinin receptor antagonist, in particular a neurokinin-1 receptor antagonist. In a preferred embodiment, the present invention provides a method for treating or preventing disturbance of appetite, disturbances of mood, or both, associated with premenstrual syndrome in a woman comprising the administration of a tachykinin receptor antagonist, in particular a neurokinin-1 receptor antagonist. The present invention is of great benefit to women who experience disturbances of mood and/or appetite prior to onset of their menstrual period, because the tachykinin receptor antagonist acts to alleviate and/or prevent such adverse premenstrual symptoms.

### DESCRIPTION OF THE INVENTION

The present invention is directed to a method for alleviating or managing symptoms associated with premenstrual syndrome in a woman comprising the administration of a tachykinin receptor antagonist, in particular an neurokinin-1 (NK-1) receptor antagonist.

In a preferred embodiment, the present invention provides a method for treating or preventing disturbance of appetite, disturbances of mood (such as depression or anxiety), or both, associated with premenstrual syndrome in a woman comprising the administration of a tachykinin receptor antagonist, in particular an NK-1 receptor antagonist.

The present invention further provides a pharmaceutical composition for alleviating or managing symptoms associated with premenstrual syndrome in a woman comprising a tachykinin receptor antagonist, in particular an NK-1 receptor antagonist, together with at least one pharmaceutically acceptable carrier or excipient.

In accordance with the present invention the tachykinin receptor antagonist is administered to a woman in a quantity sufficient to reduce, ameliorate, manage or prevent the mood and/or appetite disturbances, and/or to suppress the weight gain, which otherwise would be observed in the individual prior to onset of menstruation.

In a further aspect of the present invention, there is provided a pharmaceutical composition for the treatment or prevention of disturbances of mood and/or appetite associated with premenstrual syndrome comprising a NK-1 receptor antagonist, together with at least one pharmaceutically acceptable carrier or excipient.

The present invention also provides the use of a NK-1 receptor antagonist for the manufacture of a medicament for alleviating or managing symptoms associated with premenstrual syndrome in a woman.

Although the present invention is useful in any female mammal suffering from premenstrual syndrome, a preferred subject is a woman.

The tachykinin receptor antagonists of use in the present invention may be any tacllykinin antagonist known from the art. Preferably, the tachykinin receptor antagonist is a neurokinin-1 (NK-1) or neurokinin-2 (NK-2) receptor antagonist, especially a neurokinin-1 (NK-1) receptor antagonist.

The tachykinin antagonist may be peptidal or non-peptidal in nature, however, the use of a non-peptidal tachykinin receptor antagonist is preferred. In addition, for convenience the use of an orally active tachykinin receptor antagonist is preferred.

In the present invention, it is preferred that the tachykinin receptor antagonist is active upon the central nervous system (CNS), such as the brain, following systemic administration, i.e. that it readily penetrates the CNS. Accordingly, a preferred tachykinin antagonist for use in the present invention is a CNS-penetrating tachykinin antagonist, especially a CNS-penetrating NK-1 antagonist. An especially preferred class of NK-1 receptor antagonist of use in the present invention are those compounds which are orally active, long acting and CNS-penetrant.

Neurokinin-1 receptor antagonists of use in the present invention are fully described, for example, in U.S. Patent Nos. 5,162,339, 5,232,929, 5,242,930, 5,373,003, 5,387,595, 5,459,270, 5,494,926, 5,496,833, 5,637,699; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913,0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications.

Particularly preferred NK-1 receptor antagonists are those described in PCT International Patent Publication No. WO 95/16679 and European Patent Publication No. 0 577 394 as compounds of formula (I):

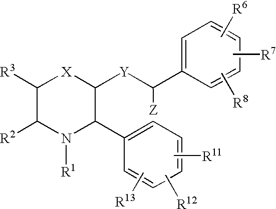

or a pharmaceutically acceptable salt thereof, wherein:

R¹ is selected from the group consisting of:

(1) hydrogen;

(2) ) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo, wherein halo is fluoro, chloro, bromo or iodo,

(h) —NR⁹R¹⁰, wherein R⁹ and R¹⁰ are independently selected from:

(i) hydrogen,

(ii) C¹⁻⁶ alkyl,

(iii) hydroxy-C₁₋₆ alkyl, and

(iv) phenyl,

(i) —NR⁹COR¹⁰,

(j) —NR9CO₂R¹⁰,

(k) —CONR⁹R¹⁰,

(l) —COR⁹,

(m) —CO₂R⁹,

(n) heterocycle, wherein the heterocycle is selected from the group consisting of:

(A) benzimidazolyl,

(B) benzofuranyl,

(C) benzothiophenyl,

(D) benzoxazolyl,

(E) furanyl,

(F) imidazolyl,

(G) indolyl,

(H) isooxazolyl,

(I) isothiazolyl,

(J) oxadiazolyl,

(K) oxazolyl,

(L) pyrazinyl,

(M) pyrazolyl,

(N) pyridyl,

(O) pyrimidyl,

(P) pyrrolyl,

(Q) quinolyl,

(R) tetrazolyl,

(S) thiadiazolyl,

(T) thiazolyl,

(U) thienyl,

(V) triazolyl,

(W) azetidinyl,

(X) 1,4-dioxanyl,

(Y) hexahydroazepinyl,

(Z) piperazinyl,

(AA) piperidinyl,

(AB) pyrrolidinyl,

(AC) tetrahydrofuranyl, and

(AD) tetrahydrothienyl,

and wherein the heterocycle is unsubstituted or substituted with one or more substituent(s) selected from:

(i) C₁₋₆ alkyl, unsubstituted or substituted with halo, —CF₃, —CF₃, —OCH₃, or phenyl,

(ii) C₁₋₆ alkoxy,

(iii) oxo,

(iv) hydroxy,

(v) thioxo,

(vi) —SR⁹,

(vii) halo,

(viii) cyano,

(ix) phenyl,

(x) trifluoromethyl,

(xi) —(CH₂)ₘ—NR⁹R¹⁰, wherein m is 0, 1 or 2,

(xii) —NR⁹COR¹⁰,

(xiii) —CONR⁹R¹⁰,

(xiv) —CO₂R⁹, and

(xv) —(CH₂)ₘ—OR⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹,

(j) —CO₂R⁹,

(k) heterocycle;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹,

(o) —CO₂R⁹;

R² and R³ are independently selected from the group consisting of:

(1) hydrogen,

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹, and

(o) —CO₂R⁹;

and the groups R¹ and R² may be joined together to form a heterocyclic ring selected from the group consisting of:

(a) pyrrolidinyl,

(b) piperidinyl,

(c) pyrrolyl,

(d) pyridinyl,

(e) imidazolyl,

(f) oxazolyl, and

(g) thiazolyl,

and wherein the heterocyclic ring is unsubstituted or substituted with one or more substituent(s) selected from:

(i) C₁₋₆alkyl,

(ii) oxo,

(iii) C₁₋₆alkoxy,

(iv) —NR⁹R¹⁰,

(v) halo, and

(vi) trifluoromethyl;

and the groups R² and R³ may be joined together to form a carbocyclic ring selected from the group consisting of:

(a) cyclopentyl,

(b) cyclohexyl,

(c) phenyl,

and wherein the carbocyclic ring is unsubstituted or substituted with one or more substituents selected from:

(i) C₁₋₆alkyl,

(ii) C₁₋₆alkoxy,

(iii) —NR⁹R¹⁰,

(iv) halo, and

(v) trifluoromethyl;

and the groups R² and R³ may be joined together to form a heterocyclic ring selected from the group consisting of:

(a) pyrrolidinyl,

(b) piperidinyl,

(c) pyrrolyl,

(d) pyridinyl,

(e) imidazolyl,

(f) furanyl,

(g) oxazolyl,

(h) thienyl, and

(i) thiazolyl,

and wherein the heterocyclic ring is unsubstituted or substituted with one or more substituent(s) selected from:

(i) C₁₋₆alkyl,

(ii) oxo,

(iii) C₁₋₆alkoxy,

(iv) —NR⁹R¹⁰,

(v) halo, and

(vi) trifluoromethyl;

R⁶, R⁷ and R⁸ are independently selected from the group consisting of:

(1) hydrogen;

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹,

(o) —CO₂R⁹;

(6) halo,

(7) —CN,

(8) —CF₃,

(9) —NO₂,

(10) —SR¹⁴, wherein R¹⁴ is hydrogen or C₁₋₅alkyl,

(11) —SOR¹⁴,

(12) —SO₂R¹⁴,

(13) NR⁹COR¹⁰,

(14) CONR⁹COR¹⁰,

(15) NR⁹R¹⁰,

(16) NR⁹CO₂R¹⁰,

(17) hydroxy,

(18) C₁₋₆alkoxy,

(19) COR⁹,

(20) CO₂R⁹,

(21) 2-pyridyl,

(22) 3-pyridyl,

(23) 4-pyridyl,

(24) 5-tetrazolyl,

(25) 2-oxazolyl, and

(26) 2-thiazolyl;

R¹¹, R¹² and R¹³ are independently selected from the definitions of R⁶, R⁷ and R⁸;

X is selected from the group consisting of:

(1) —O—;

(2) —S—,

(3) —SO—, and

(4) —SO₂—;

Y is selected from the group consisting of:

(1) a single bond,

(2) —O—,

(3) —S—,

(4) —CO—,

(5) —CH₂—,

(6) —CHR¹⁵—, and

(7) —CR¹⁵R¹⁶—, wherein R¹⁵ and R¹⁶ are independently selected from the group consisting of:

(a) C₁₋₆ alkyl, unsubstituted or substituted with one or more of the substituents selected from:

(i) hydroxy,

(ii) oxo,

(iii) C₁₋₆ alkoxy,

(iv) phenyl-C₁₋₃ alkoxy,

(v) phenyl,

(vi) —CN,

(vii) halo,

(viii) —NR⁹R¹⁰,

(ix) —NR⁹COR¹⁰,

(x) —NR⁹CO₂R¹⁰,

(xi) —CONR⁹R¹⁰,

(xii) —COR⁹, and

(xiii) —CO₂R⁹;

(b) phenyl, unsubstituted or substituted with one or more of the substituent(s) selected from:

(i) hydroxy,

(ii) C₁₋₆ alkoxy,

(iii) C₁₋₆ alkyl,

(iv) C₂₋₅ alkenyl,

(v) halo,

(vi) —CN,

(vii) —NO₂,

(viii) —CF₃,

(ix) —(CH₂)ₘ—NR⁹R¹⁰,

(x) —NR⁹COR¹⁰,

(xi) —NR⁹CO₂R¹⁰,

(xii) —CONR⁹R¹⁰,

(xiii) —CO₂NR⁹R¹⁰,

(xiv) —COR⁹, and

(xv) —CO₂R⁹; and

Z is C₁₋₆ alkyl.

Particularly preferred compounds of formula (I) include:

4-(3-(1,2,4-triazolo)_{methyl)-}2(S)-(3,5-bis(trifluoro-methyl)benzyloxy)-3(S)-phenyl-morpholine;

4-(3-(1,2,4-triazolo)methyl)-2(S)-(3,5-bis(trifluoro-methyl)benzyloxy)-3(R)-phenyl-morpholine;

4-(3-(5-oxo-1H,4H-1,2,4-triazolo)methyl)-2(S)-(3,5-bis(trifluoromethyl)benzyloxy)-3(S)-phenyl-morpholine; and

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine; or a pharmaceutically acceptable salt thereof.

An especially preferred compound of formula (I) is

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methylmorpholine; or a pharmaceutically acceptable salt thereof.

Further preferred NK-1 receptor antagonists are those described in PCT International Patent Publication No. WO 95/18124 as compounds of formula (II):

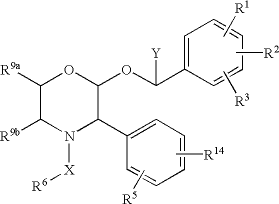

or a pharmaceutically acceptable salt or prodrug thereof, wherein

R¹ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a} and R^{b} each independently represent hydrogen or C₁₋₄alkyl;

R² is hydrogen, halogen, C₁₋₆alkyl, C₁₋₆alkoxy substituted by C₁₄alkoxy or CF₃;

R³ is hydrogen, halogen or CF₃;

R⁴ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, where R^{a} and R^{b} each independently represent hydrogen or C₁₋₄alkyl;

R⁵ is hydrogen, halogen, C₁₋₆alkyl, C₁₋₆alkoxy substituted by C₁₋₄alkoxy or CF₃;

R⁶ is a 5-membered or 6-membered heterocyclic ring containing 2 or 3 nitrogen atoms optionally substituted by ═O, ═S or a C₁₋₄alkyl group, and optionally substituted by a group of the formula ZNR⁷R⁸ where

Z is C₁₋₆alkylene or C₃₋₆cycloalkylene;

R⁷ is hydrogen, C₁₋₄alkyl, C₃₋₇cycloalkyl or C₃₋₇cycloalkylC₁₋₄alkyl, or C₂₋₄alkyl substituted by C₂₋₄alkoxy or hydroxyl;

R⁸ is hydrogen, C₁₋₄alkyl, C₃₋₇cycloalkyl or C₃₋₇cycloalkylC₁₋₄alkyl, or C₂₋₄alkyl substituted by one or two substituents selected from C₁₋₄alkoxy, hydroxyl or a 4, 5 or 6 membered heteroaliphatic ring containing one or two heteroatoms selected from N, O and S;

or R⁷, R⁸ and the nitrogen atom to which they are attached form a heteroaliphatic ring of 4 to 7 ring atoms, optionally substituted by a hydroxy group, and optionally containing a double bond, which ring may optionally contain an oxygen or sulphur ring atom, a group S(O) or S(O)₂ or a second nitrogen atom which will be part of a NH or NRC moiety where R^{c} is C₁₋₄alkyl optionally substituted by hydroxy or C₁₋₄alkoxy;

or R⁷, R⁸ and the nitrogen atom to which they are attached form a non-aromatic azabicyclic ring system of 6 to 12 ring atoms; or Z, R⁷ and the nitrogen atom to which they are attached form a heteroaliphatic ring of 4 to 7 ring atoms which may optionally contain an oxygen ring atom;

R^{9a} and R^{9b} are each independently hydrogen or C₁₋₄alkyl, or R^{9a} and R^{9b} are joined so, together with the carbon atoms to which they are attached, there is formed a C₅₋₇ ring;

X is an alkylene chain of 1 to 4 carbon atoms optionally substituted by oxo; and

Y is a C₁₋₄alkyl group optionally substituted by a hydroxyl group; with the proviso that if Y is C₁₋₄alkyl, R⁶ is substituted at least by a group of formula ZNR⁷R⁸ as defined above.

Particularly preferred compounds of formula (II) include:

2-(R)-(1-(R)-(3,5-bis(trifluoromethyl)phenyl)ethoxy)-4-(5-(dimethylamino)methyl-1,2,3-triazol-4-yl)methyl-3-(S)-phenylmorpholine;

(1-(R)-(3,5-bis(trifluoromethyl)phenyl)ethoxy)-4-((dimethylamino-methyl)-1,2,3-triazol-4-yl)methyl)-3-(S)-(4-fluorophenyl)morpholine;

2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine; or a pharmaceutically acceptable salt thereof.

Further preferred NK-1 receptor antagonists are those described in PCT International Patent Publication No. WO 95/23798 as compounds of formula (III):

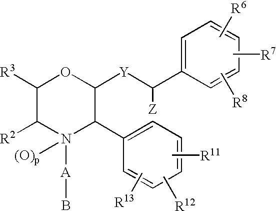

or a pharmaceutically acceptable salt thereof, wherein:

R² and R³ are independently selected from the group consisting of:

(1) hydrogen,

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰, wherein R⁹ and R¹⁰ are independently selected from:

(i) hydrogen,

(ii) C₁₋₆ alkyl,

(iii) hydroxy-C₁₋₆ alkyl, and

(iv) phenyl,

(i) —NR⁹COR¹⁰,

(j) —NR⁹CO₂R¹⁰,

(k) —CONR⁹R¹⁰,

(l) —COR⁹, and

(m) —CO₂R₉;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹, and

(o) —CO₂R⁹;

and, alternatively, the groups R² and R³ are joined together to form a carbocyclic ring selected from the group consisting of:

(a) cyclopentyl,

(b) cyclohexyl,

(c) phenyl,

and wherein the carbocyclic ring is unsubstituted or substituted with one or more substituents selected from:

(i) C₁₋₆alkyl,

(ii) C₁₋₆alkoxy,

(iii) —NR⁹R¹⁰,

(iv) halo, and

v) trifluoromethyl;

and, alternatively, the groups R² and R³ are joined together to form a heterocyclic ring selected from the group consisting of:

(a) pyrrolidinyl,

(b) piperidinyl,

(c) pyrrolyl,

(d) pyridinyl,

(e) imidazolyl,

(f) furanyl,

(g) oxazolyl,

(h) thienyl, and

(i) thiazolyl,

and wherein the heterocyclic ring is unsubstituted or substituted with one or more substituent(s) selected from:

(i) C₁₋₆alkyl,

(ii) oxo,

(iii) C₁₋₆alkoxy,

(iv) —NR⁹R¹⁰,

(v) halo, and

(vi) trifluoromethyl;

R⁶, R⁷ and R⁸ are independently selected from the group consisting of:

(1) hydrogen;

(2) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(3) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹;

(4) C₂₋₆ alkynyl;

(5) phenyl, unsubstituted or substituted with one or more of the substituent(s) selected from:

(a) hydroxy,

(b) C₁₋₆ alkoxy,

(c) C₁₋₆ alkyl,

(d) C₂₋₅ alkenyl,

(e) halo,

(f) —CN,

(g) —NO₂,

(h) —CF₃,

(i) —(CH₂)ₘ—NR⁹R¹⁰,

(j) —NR⁹COR¹⁰,

(k) —NR⁹CO₂R¹⁰,

(l) —CONR⁹R¹⁰,

(m) —CO₂NR⁹R¹⁰,

(n) —COR⁹, and

(o) —CO₂R⁹;

(6) halo,

(7) —CN,

(8) —CF₃,

(9) —NO₂,

(10) —SR¹⁴, wherein R¹⁴ is hydrogen or C₁₋₅alkyl,

(11) —SOR¹⁴,

(12) —SO₂R¹⁴,

(13) NR⁹COR¹⁰,

(14) CONR⁹COR¹⁰,

(15) NR⁹R¹⁰,

(16) NR⁹CO₂R¹⁰,

(17) hydroxy,

(18) C₁₋₆alkoxy,

(19) COR⁹,

(20) CO₂R⁹,

(21) 2-pyridyl,

(22) 3-pyridyl,

(23) 4-pyridyl,

(24) 5-tetrazolyl,

(25) 2-oxazolyl, and

(26) 2-thiazolyl;

R¹¹, R¹² and R¹³ are independently selected from the definitions of R⁶, R⁷ and R⁸, or —OX;

A is selected from the group consisting of:

(1) C₁₋₆ alkyl, unsubstituted or substituted with one or more of the substituents selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —NR⁹R¹⁰,

(i) —NR⁹COR¹⁰,

(j) —NR⁹CO₂R¹⁰,

(k) —CONR⁹R¹⁰,

(l) —COR⁹, and

(m) —CO₂R⁹;

(2) C₂₋₆ alkenyl, unsubstituted or substituted with one or more of the substituent(s) selected from:

(a) hydroxy,

(b) oxo,

(c) C₁₋₆ alkoxy,

(d) phenyl-C₁₋₃ alkoxy,

(e) phenyl,

(f) —CN,

(g) halo,

(h) —CONR⁹R¹⁰,

(i) —COR⁹, and

(j) —CO₂R⁹; and

(3) C₂₋₆ alkynyl;

B is a heterocycle, wherein the heterocycle is selected from the group consisting of:

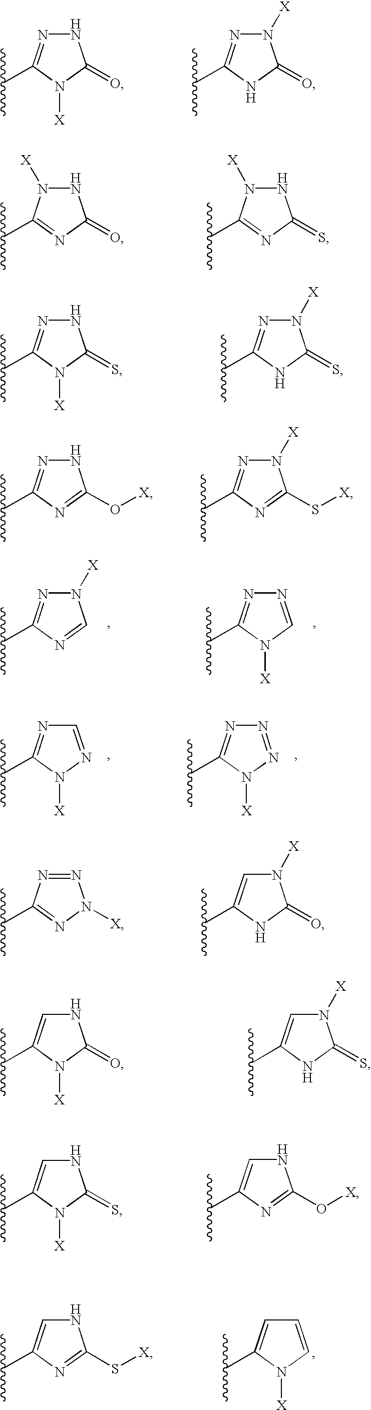

and wherein the heterocycle is substituted in addition to —X with one or more substituent(s) selected from:

(i) hydrogen;

(ii) C₁₋₆ alkyl, unsubstituted or substituted with halo, —CF₃, —OCH₃, or phenyl,

(iii) C₁₋₆ alkoxy,

(iv) oxo,

(v) hydroxy,

(vi) thioxo,

(vii) —SR⁹,

(viii) halo,

(ix) cyano,

(x) phenyl,

(xi) trifluoromethyl,

(xii) —(CH₂)ₘ—NR⁹R¹⁰, wherein m is 0, 1 or 2,

(xiii) —NR⁹COR¹⁰,

(xiv) —CONR⁹R¹⁰,

(xv) —CO₂R⁹, and

(xvi) —(CH₂)ₘ—OR⁹;

p is 0 or 1;

X is selected from:

(a) —PO(OH)O⁻.M⁺, wherein M⁺ is a pharmaceutically acceptable monovalent counterion,

(b) —PO(O⁻)₂.2M⁺,

(c) —PO(O—)₂.D²⁺, wherein D²+ is a pharmaceutically acceptable divalent counterion,

(d) —CH(R⁴)—PO(OH)O⁻.M⁺, wherein R⁴ is hydrogen or C₁₋₃ alkyl,

(e) —CH(R⁴)—PO(O⁻)₂.2M⁺,

(f) —CH(R⁴)—PO(O⁻)₂.D²⁺,

(g) —SO₃⁻.M+,

(h) —CH(R⁴)—SO₃⁻.M⁺,

(i) —CO—CH₂CH₂—CO₂⁻.M⁺,

(j) —CH(CH₃)—O—CO—R⁵, wherein R⁵ is selected from the group consisting of:

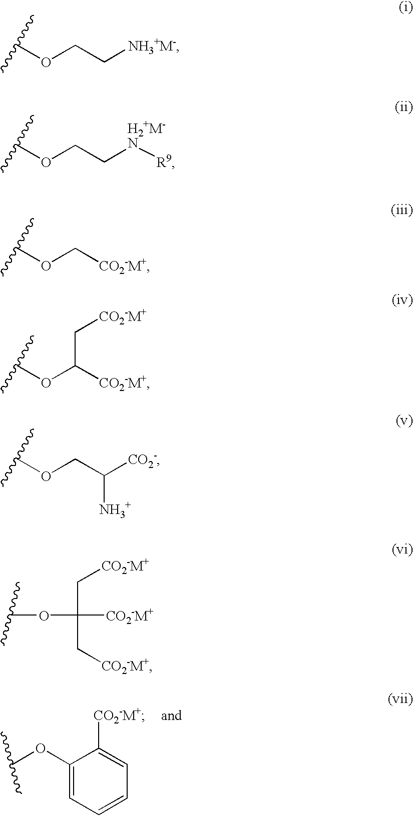

(k) hydrogen, with the proviso that if p is 0 and none of R¹¹, R¹² or R¹³ are —OX, then X is other than hydrogen;

Y is selected from the group consisting of:

(1) a single bond,

(2) —O—,

(3) —S—,

(4) —CO—,

(5) —CH₂—,

(6) —CHR¹⁵—, and

(7) —CR¹⁵R¹⁶—, wherein R¹⁵ and R¹⁶ are independently selected from the group consisting of:

(a) C₁₋₆ alkyl, unsubstituted or substituted with one or more of the substituents selected from:

(i) hydroxy,

(ii) oxo,

(iii) C₁₋₆ alkoxy,

(iv) phenyl-C₁₋₃ alkoxy,

(v) phenyl,

(vi) —CN,

(vii) halo,

(viii) —NR⁹R¹⁰,

(ix) —NR⁹COR¹⁰,

(x) —NR⁹CO₂R¹⁰,

(xi) —CONR⁹R¹⁰,

(xii) —COR⁹, and

(xiii) —CO₂R⁹;

(b) phenyl, unsubstituted or substituted with one or more of the substituent(s) selected from:

(i) hydroxy,

(ii) C₁₋₆ alkoxy,

(iii) C₁₋₆ alkyl,

(iv) C₂₋₅ alkenyl,

(v) halo,

(vi) —CN,

(vii) —NO₂,

(viii) —CF₃,

(ix) —(CH₂)ₘ—NR⁹R¹⁰,

(x) —NR⁹COR¹⁰,

(xi) —NR⁹CO₂R¹⁰,

(xii) —CONR⁹R¹⁰,

(xiii) —CO₂NR⁹R¹⁰,

(xiv) —COR⁹, and

(xv) —CO₂R⁹;

Z is selected from:

(1) hydrogen,

(2) C₁₋₆ alkyl, and

(3) hydroxy, with the proviso that if Y is —O—, Z is other than hydroxy, or if Y is —CHR¹⁵—, then Z and R¹⁵ are optionally joined together to form a double bond.

A particularly preferred compound of formula (III) is 2-(R)-(1-(R)-(3,5-bis(trifluoromethyl)phenyl)ethoxy)-3-(S)-(4-fluoro)-phenyl-4-(3-(1-monophosphoryl-5-oxo-1H-1,2,4-triazolo)methyl)morpholine, or a pharmaceutically acceptable salt thereof. In particular, the bis(N-methyl-D-glucamine) salt is preferred.

Further preferred NK-1 receptor antagonists are those described in European Patent Specification No. WO 96/05181, i.e. compounds of formula (IV):

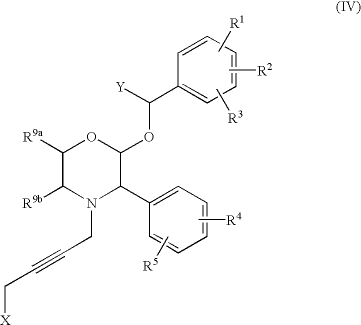

wherein:

X is a group of the formula NR⁶R⁷ or a C- or N-linked imidazolyl ring;

Y is hydrogen or C₁₋₄alkyl optionally substituted by a hydroxy group;

R¹ is hydrogen, halogen, C₁₋₆alkyl, C₁₋₆alkoxy, CF₃, NO₂, CN, SR^{a}, SOR^{a}, SO₂R^{a}, CO₂R^{a}, CONR^{a}R^{b}, C₂₋₆alkenyl, C₂₋₆alkynyl or C₁₋₄alkyl substituted by C₁₋₄alkoxy, wherein R^{a} and R^{b} each independently represent hydrogen or C₁₋₄alkyl;

R² is hydrogen, halogen, C₁₋₆alkyl, C₁₋₆alkoxy substituted by C₁₋₄alkoxy or CF₃;

R³ is hydrogen, halogen or CF₃;

R⁴ is hydrogen, halogen, C₁₋₆alkyl, C₁₋₆alkoxy, hydroxy, CF₃, NO₂, CN, SR^{a}, SOR^{a}, SO₂R^{a}, CO₂R^{a}, CONR^{a}R^{b}, C₂₋₆alkenyl, C₂₋₆alkynyl C₁₋₄alkyl substituted by C₁₋₄alkoxy, wherein R^{a} and R^{b} are as previously defined;

R⁵ is hydrogen, halogen, C₁₋₆alkyl, C₁₋₆alkoxy substituted by C₁₋₄alkoxy or CF₃;

R⁶ is hydrogen, C₁₋₆alkyl, C₃₋₇cycloalkyl, C₃₋₇cycloalkylC₁₋₄alkyl, phenyl, or C₂₋₄alkyl substituted by C₁₋₄alkoxy or hydroxy;

R⁷ is hydrogen, C₁₋₆alkyl, C₃₋₇cycloalkyl, C₃₋₇cycloalkylC₁₋₄alkyl, phenyl, or C₂₋₄alkyl substituted by one or two substituents selected from C₁₋₄alkoxy, hydroxy or a 4, 5 or 6 membered heteroaliphatic ring containing one or two heteroatoms selected from N, O and S;

or R⁶ and R⁷, together with the nitrogen atom to which they are attached, form a saturated or partially saturated heterocyclic ring of 4 to 7 ring atoms, which ring may optionally contain in the ring one oxygen or sulphur atom or a group selected from NR⁸, S(O) or S(O)₂ and which ring may be optionally substituted by one or two groups selected from hydroxyC₁₋₄alkyl, C₁₋₄alkoxyC₁₋₄alkyl, oxo, COR^{a} or CO₂R^{a} where R^{a} is as previously defined;

or R⁶ and R⁷ together with the nitrogen atom to which they are attached, form a non-aromatic azabicyclic ring system of 6 to 12 ring atoms;

R⁸ is hydrogen, C₁₋₄alkyl, hydroxyC₁₋₄alkyl or C₁₋₄alkoxyC₁₋₄alkyl; and

R^{9a} and R^{9b} are each independently hydrogen or C₁₋₄alkyl, or R^{9a} and R^{9b} are joined so, together with the carbon atoms to which they are attached, there is formed a C₅₋₇ ring;

and pharmaceutically acceptable salts thereof.

Specific compounds of formula (IV) of use in the present invention include:

2-(R)-(1-(R)-(3,5-bis(trifluoromethyl)phenyl)ethoxy)-3-(S)-(4-fluorophenyl)-4-(4-morpholinobut-2-yn-yl)morpholine;

2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;

4-(4-azetidinylbut-2-yn-yl)-2-(R)-(1-(R)-(3,5-bis(trifluoromethyl)phenyl)ethoxy)-3-(S)-(4-fluorophenyl)morpholine;

2-(R)-(1-(R)-(3,5-bis(trifluoromethyl)phenyl)ethoxy)-3-(S)-(4-fluorophenyl)-4-(4-imidazolylbut-2-yn-yl)morpholine;

2-(R)-(1-(R)-(3,5-bis(trifluoromethyl)phenyl)ethoxy)-3-(S)-(4-fluorophenyl)-4-(4-(N-methylpiperazinyl)but-2-yn-yl)morpholine;

4-(4-bis(2-methoxyethyl)aminobut-2-yn-yl)-2-(R)-(1-(R)-(3,5-bis(trifluoromethyl)phenyl)ethoxy)-3-(S)-(4-fluorophenyl)morpholine;

2-(R)-(1-(R)-(3,5-bis(trifluoromethyl)phenyl)ethoxy)-3-(S)-(4-fluorophenyl)-4-(4-pyrrolidinobut-2-yn-yl)morpholine;

3-(S)-(4-fluorophenyl)-2-(R)-(1-(R)-(3-fluoro-5-(trifluoromethyl)phenyl)ethoxy)-4-(4-morpholinobut-2-yn-yl)morpholine;

3-(S)-(4-fluorophenyl)-4-(4-morpholinobut-2-yn-yl)-2-(R)-( 1-(R)-(3-(trifluoromethyl)phenyl)ethoxy)morpholine;

4-(4-azetidinylbut-2-yn-yl)-3-(S)-(4-fluorophenyl)-2-(R)-(1-(R)-(3-(trifluoromethyl)phenyl)ethoxy)morpholine;

2-(R)-(1-(R)-(3,5-bis(trifluoromethyl)phenyl)ethoxy)-4-(4-(N-(2-methoxyethyl)-N-methyl)aminobut-2-yn-yl)-3-(S)-phenylmorpholine;

2-(R)-(1-(R)-(3,5-bis(trifluoromethyl)phenyl)ethoxy)-4-(4-(N-cyclopropyl-N-(2-methoxyethyl)amino)but-2-yn-yl)-3-(S)-phenylmorpholine;

2-(R)-(1-(R)-(3,5-bis(trifluoromethyl)phenyl)ethoxy)-4-(4-(N-isopropyl-N-(2-methoxyethyl)amino)but-2-yn-yl)-3-(S)-phenylmorpholine;

4-(4-(N,N-dimethylamino)but-2-yn-yl)-3-(S)-(4-fluorophenyl)-2-(R)-(1-(S)-(3-fluoro-5-(trifluorometbyl)phenyl-2-hydroxyethoxy)morpholine;

4-(4-azetidinylbut-2yn-yl)-3-(S)-(4-fluorophenyl)-2-(R)-(1-(S)-(3-fluoro-5-(trifluoromethyl)phenyl)-2-hydroxyethoxy)morpholine;

2-(R)-(1-(S)-(3,5-bis(trifluoromethy)phenyl)-2-hydroxyethoxy)-4-(4-(N,N-dimethylamino)but-2-yn-yl)-3-(S)-(4-fluorophenyl)morpholine;

4-(4-azetidinylbut-2-yn-yl)-2-(R)-(1-(S)-(3,5-bis(trifluoromethyl)phenyl-2-hydroxyethoxy)-3-(S)-(4-fluorophenyl)morpholine;

4-(4-N-bis(2-methoxy)ethyl-N-methylamino)but-2-yn-yl)-2-(R)-(l-(R)-(3,5-bis(trifluoromethyl)phenyl)ethoxy)-3-(S)-(4-fluorophenyl)morpholine;

2-(R)-(1-(R)-(3,5-bis(trifluoromethyl)phenyl)ethoxy)-3-(S)-(4-fluorophenyl)-4-(4-(2-(S)-(methoxymethyl)pyrrolidino)but-2-yn-yl)morpholine;

4-(4-(7-azabicyclo [2.2.]heptano)but-2-yn-yl)-2-(R)-(1-(R)-(3,5-bis(trifluoromethyl)phenyl)ethoxy)-3-(S)-(4-fluorophenyl)morpholine;

2-(R)-(1-(R)-(3,5-bis(trifluoromethyl)phenyl)ethoxy)-4-(4-diisopropylaminobut-2-yn-yl)-3-(S)-(4-fluorophenyl)morpholine;

2-(R)-(1-(R)-(3-fluoro-5-(trifluoromethyl)phenyl)ethoxy)-4-(4-(2-(S)-(methoxymethyl)pyrrolidino)but-2-yn-yl)-3-(S)-phenylmorpholine;

2-(R)-(1-(R)-(3,5-bis(trifluoromethyl)phenyl)ethoxy)-3-(S)-(4-fluorophenyl)-4-(4-(2-(S)-(hydroxymethyl)pyrrolidino)but-2-yn-yl)morpholine;

and pharmaceutically acceptable salts thereof.

Further preferred NK-1 receptor antagonists are those described in European Patent Publication No. 0 436 334 as compounds of formula (V):

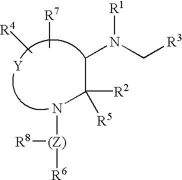

or a pharmaceutically acceptable salt thereof, wherein

Y is (CH₂)ₙ wherein n is an integer from 1 to 4, and wherein any one of the carbon-carbon single bonds in said (CH₂)ₙ may optionally be replaced by a carbon-carbon double bond, and wherein any one of the carbon atoms of said (CH₂)ₙ may optionally be substituted with R⁴, and wherein any one of the carbon atoms of said (CH₂)ₙ may optionally be substituted with R⁷;

Z is (CH₂)ₘ wherein m is an integer from 0 to 6, and wherein any one of the carbon-carbon single bonds of (CH₂)ₘ may optionally be replaced by a carbon-carbon double bond or a carbon-carbon triple bond, and any one of the carbon atoms of said (CH₂)ₘ may optionally be substituted with R⁸;

R₁ is hydrogen or C₁₋₈alkyl optionally substituted with hydroxy, C₁₋₄alkoxy or fluoro;

R² is a radical selected from hydrogen, C₁₋₆ straight or branched alkyl, C₃₋₇cycloalkyl wherein one of the CH2 groups in said cycloalkyl may optionally be replaced by NH, oxygen or sulphur; aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; phenyl-C₂₋₆alkyl, benzhydryl and benzyl, wherein each of said aryl and heteroaryl groups and the phenyl moieties of said benzyl, phenyl —C₂₋₆alkyl and benzhydryl may optionally be substituted with one or more substituents independently selected from halo, nitro, C₁₋₆alkyl, C₁₋₆alkoxy, trifluoromethyl, amino, C₁₋₆alkylamino, C₁₋₆alkyl-O—CO, C₁₋₆alkyl-O—CO—C₁₋₆alkyl, C₁₋₆alkyl-CO—O, C₁₋₆alkyl-CO—C₁₋₆alkyl-O—, C₁₋₆alkyl-CO, C₁₋₆alkyl-CO—C₁₋₆alkyl-, di-C₁₋₆alkylamino, —CONH—C₁₋₆alkyl, C₁₋₆alkyl-CO—NH—C₁₋₆alkyl, —NHCOH and —NHCO—C₁₋₆alkyl; and wherein one of the phenyl moieties of said benzhydryl may optionally be replaced by naphthyl, thienyl, furyl or pyridyl;

R⁵ is hydrogen, phenyl or C₁₋₆alkyl;

or R² and R⁵ together with the carbon to which they are attached, form a saturated ring having from 3 to 7 carbon atoms wherein one of the CH₂ groups in said ring may optionally be replaced by oxygen, NH or sulfur;

R³ is aryl selected from phenyl and naphthyl; heteroaryl selected from indanyl, thienyl, furyl, pyridyl, thiazolyl, isothiazolyl, oxazolyl, isoxazolyl, triazolyl, tetrazolyl and quinolyl; and cycloalkyl having 3 to 7 carbon atoms wherein one of the (CH₂) groups in said cycloalkyl may optionally be replaced by NH, oxygen or sulphur;

wherein each of said aryl and heteroaryl groups may optionally be substituted with one or more substituents, and said C₃₋₇cycloalkyl may optionally be substituted with one or two substituents, each of said substituents being independently selected from halo, nitro, C₁₋₆alkyl, C₁₋₆alkoxy, trifluoromethyl, amino, C₁₋₆alkylamino, —CO—NH—C₁₋₆alkyl, C₁₋₆alkyl-CO—NH—C₁₋₆alkyl, —NHCOH and —NH CO—C₁₋₆alkyl;

R⁴ and R⁷ are each independently selected from hydroxy, halogen, halo, amino, oxo, cyano, methylene, hydroxymethyl, halomethyl, C₁₋₆alkylamino, di-C₁₋₆alkylamino, C₁₋₆alkoxy, C₁₋₆alkyl-O—CO, C₁₋₆alkyl-O—CO—C₁₋₆alkyl, C₁₋₆alkyl-CO—O, C₁₋₆alkyl-CO—C₁₋₆alkyl-O—, C₁₋₆alkyl-CO—, C₁₋₆alkyl-CO—C₁₋₆alkyl, and the radicals set forth in the definition of R²;

R⁶ is —NHCOR⁹, —NHCH₂R⁹, SO₂R⁸ or one of the radicals set forth in any of the definitions of R², R⁴ and R⁷;

R⁸ is oximino (═NOH) or one of the radicals set forth in any of the definitions of R², R⁴ and R⁷;

R⁹ is C₁₋₆alkyl, hydrogen, phenyl or phenylC₁₋₆alkyl;

with the proviso that (a) when m is 0, R⁸ is absent, (b) when R⁴, R⁶, R⁷ or R⁸ is as defined in R², it cannot form together with the carbon to which it is attached, a ring with R⁵, and (c) when R⁴ and R⁷ are attached to the same carbon atom, then either each of R⁴ and R⁷ is independently selected from hydrogen, fluoro and C₁₋₆alkyl, or R⁴ and R⁷, together with the carbon to which they are attached, for a C₃₋₆ saturated carbocyclic ring that forms a spiro compound with the nitrogen-containing ring to which they are attached.

A particularly preferred compound of formula (V) is (2S,3S)-cis-3-(2-methoxybenzylamaino)-2-phenylpiperidine; or a pharmaceutically acceptable salt thereof.

Another class of NK-1 receptor antagonists is as disclosed in PCT International Patent Publication No. WO 93/21155 as compounds of formula (VI):

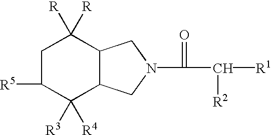

or a pharmaceutically acceptable salt thereof, wherein

radicals R are phenyl radicals optionally 2-or 3-substituted by a halogen atom or a methyl radical;

R¹ is optionally substituted phenyl, cyclohexadienyl, naphthyl, indenyl or optionally substituted heterocycle;

R² is H, halogen, OH, alkyl, aminoalkyl, alkylaminoalkyl, dialkylaminoalkyl, alkyloxy, alkylthio, acyloxy, carboxy, optionally substituted alkyloxycarbonyl, benzyloxycarbonyl, amino or acylamino;

R³ is optionally 2-substituted phenyl;

R⁴ is OH or fluorine when R⁵ is H;

or R⁴ and R⁵ are OH;

or R⁴ and R⁵ together form a bond.

A particularly preferred compound of formula (VI) is (3aS,4S,7aS)-7,7-diphenyl-4-(2-methoxyphenyl)-2-[(2S)-(2-methoxy-phenyl)propionyl] perhydroisoindol-4-ol; or a pharmaceutically acceptable salt thereof.

Another class of NK-1 receptor antagonists of use in the present invention is that described in European Patent Publication No. 0 591 040 as compounds of formula (VII):

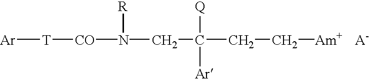

wherein

Ar represents an optionally substituted mono-, di-or tricyclic aromatic or heteroaromatic group;

T represents a bond, a hydroxymethylene group, a C₁₋₄alkoxymethylene group or a C₁₋₅alkylene group;

Ar′ represents a phenyl group which is unsubstituted or substituted by one or more substituents selected from halogen, preferably chlorine or fluorine, trifluoromethyl, C₁₋₄alkoxy, C₁₋₄alkyl where the said substituents may be the same or different; a thienyl group; a benzothienyl group; a naphthyl group; or an indolyl group;

R represents hydrogen, C₁₋₄alkyl, —C₁₋₄alkoxyC₁₋₄alkyl, or -C₂₋₄alkanoyloxyC₂₋₄alkyl;

Q represents hydrogen;

or Q and R together form a 1,2-ethylene, 1,3-propylene or 1,4-butylene group;

Am⁺ represents the radical

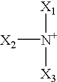

in which

X₁, X₂ and X₃, together with the nitrogen atom to which they are attached, form an azabicyclic or azatricyclic ring system optionally substituted by a phenyl or benzyl group; and

A⁻ represents a pharmaceutically acceptable anion.

A particularly preferred compound of formula (VII) is (+) 1-[2-[3-(3,4-dichlorophenyl)-1-[(3-isopropoxyphenyl)acetyl]-3-piperidinyl]ethyl]-4-phenyl-1-azabicyclo[2,2,2]octane; or a pharmaceutically acceptable salt, especially the chloride, thereof.

Another class of NK-1 receptor antagonists of use in the present invention is that described in European Patent Publication No. 0 532 456 as compounds of formula (VIII):

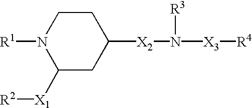

or a pharmaceutically acceptable salt thereof, wherein

R¹ represents an optionally substituted aralkyl, aryloxyalkyl, heteroaralkyl, aroyl, heteroaroyl, cycloalkylcarbonyl, aralka noyl, heteroarylalkanoyl, aralkoxycarbonyl or arylcarbamoyl group or the acyl group of an (-amino acid optionally N-substituted by a lower alkanoyl or carbamoyl-lower alkanoyl group;

R² represents cycloalkyl or an optionally substituted aryl or heteroaryl group;

R³ represents hydrogen, alkyl, carbamoyl or an alkanoyl or alkenoyl group optionally substituted by carboxy or esterified or amidated carboxy;

R⁴ represents an optionally substituted aryl group or an optionally partially saturated heteroaryl group;

X₁ represents methylene, ethylene, a bond, an optionally ketalised carbonyl group or an optionally etherified hydroxymethylene group;

X₂ represents alkylene, carbonyl or a bond; and

X₃ represents carbonyl, oxo-lower alkyl, oxo(aza)-lower alkyl, or an alkyl group optionally substituted by phenyl, hydroxymethyl, optionally esterified or amidated carboxy, or (in other than the (-position) hydroxy.

A particularly preferred compound of formula (VIII) is (2R*, 4S*)-2-benzyl-1-(3,5-dimethylbenzoyl)-N-(4-quinolinylmethyl)-4-piperidineamine; or a pharmaceutically acceptable salt thereof.

Another class of NK-1 receptor antagonists of use in the present invention is that described in European Patent Publication No. 0 443 132 as compounds of formula (IX):

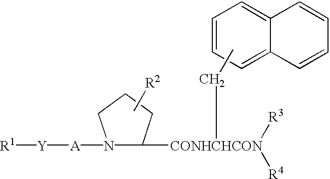

or a pharmaceutically acceptable salt thereof, wherein wherein R¹ is aryl, or a group of the formula:

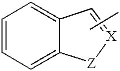

or a pharmaceutically acceptable salt thereof, wherein

X is CH or N; and

Z is O or N—R⁵, in which R⁵ is hydrogen or lower alkyl;

R² is hydroxy or lower alkoxy;

R³ is hydrogen or optionally substituted lower alkyl;

R⁴ is optionally substituted ar(lower)alkyl;

A is carbonyl or sulfonyl; and

Y is a bond or lower alkenylene.

A particularly preferred compound of formula (IX) is the compound of formula (IXa)

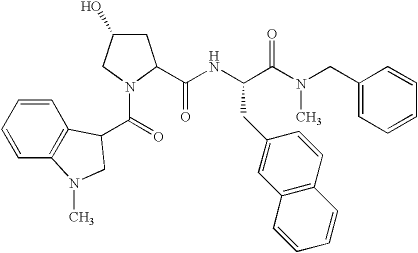

Another class of NK-1 receptor antagonists of use in the present invention is that described in PCT International Patent Publication No. WO 92/17449 as compounds of the formula (X):

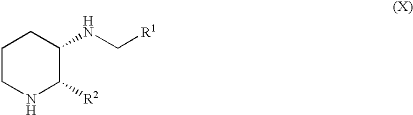

or a pharmaceutically acceptable salt thereof, wherein

R¹ is aryl selected from indanyl, phenyl and naphthyl; heteroaryl selected from thienyl, furyl, pyridyl and quinolyl; and cycloalkyl having 3 to 7 carbon atoms, wherein one of said carbon atoms may optionally be replaced by nitrogen, oxygen or sulfur; wherein each of said aryl and heteroaryl groups may optionally be substituted with one or more substituents, and said C₃₋₇cycloalkyl may optionally be substituted with one or two substituents, said substituents being independently selected from chloro, fluoro, bromo, iodo, nitro, C₁₋₁₀alkyl optionally substituted with from one to three fluoro groups, C₁₋₁₀alkoxy optionally substituted with from one to three fluoro groups, amino, C₁₋₁₀alkyl-S—, C₁₋₁₀alkyl-S(O)—, C₁₋₁₀alkyl-SO₂—, phenyl, phenoxy, C₁₋₁₀alkyl-SO₂NH—, C₁₋₁₀alkyl-SO₂NH—C₁₋₁₀alkyl—, C₁₋₁₀alkylamino-diC₁₋₁₀alkyl-, cyano, hydroxy, cycloalkoxy having 3 to 7 carbon atoms, C₁₋₆alkylamino, C₁₋₆dialkylamino, HC(O)NH— and C₁₋₁₀alkyl-C(O)NH—; and

R² is thienyl, benzhydryl, naphthyl or phenyl optionally substituted with from one to three substituents independently selected from chloro, bromo, fluoro, iodo, cycloalkoxy having 3 to 7 carbon atoms, C₁₋₁₀alkyl optionally substituted with from one to three fluoro groups and C₁₋₁₀alkoxy optionally substituted with from one to three fluoro groups.

A particularly preferred compound of formula (X) is (2S,3S)-3-(2-methoxy-5-trifluoromethoxybenzyl)-amino-2-phenylpiperidine; or a pharmaceutically acceptable salt thereof.

Another class of NK-1 receptor antagonists of use in the present invention is that described in PCT International Patent Publication No. WO 95/08549 as compounds of formula (XI):

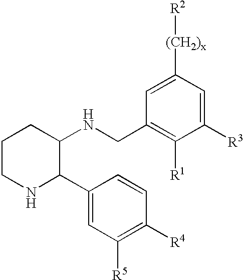

wherein

R¹ is a C₁₋₄alkoxy group;

R² is

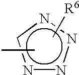

R³ is a hydrogen or halogen atom;

R⁴ and R⁵ may each independently represent a hydrogen or halogen atom, or a C₁₋₄ alkyl, C₁₋₄ alkoxy or trifluoromethyl group;

R⁶ is a hydrogen atom, a C₁₋₄ alkyl, (CH₂)ₘ cyclopropyl, —S(O)ₙC₁₋₄ alkyl, phenyl, NR⁷R⁸, CH₂C(O)CF₃ or trifluoromethyl group;

R⁷ and R⁸ may each independently represent a hydrogen atom, or a C₁₋₄ alkyl or acyl group;

x represents zero or 1;

n represents zero, 1 or 2;

m represents zero or 1;

and pharmaceutically acceptable salts and solvates thereof.

A particularly preferred compound of formula (XI) is [2-methoxy-5-(5-trifluoromethyl-tetrazol-1-yl)-benzyl]-(2S-phenyl-piperidin-3S-yl)-amine; or a pharmaceutically acceptable salt thereof.

Another class of NK-1 receptor antagonists of use in the present invention is that described in PCT International Patent Publication No. WO 97/49710 as compounds of formula (XII):

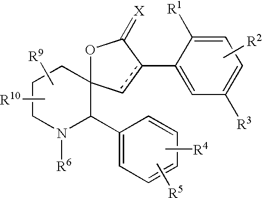

wherein R¹, R², R³, R⁴, R⁵, R⁶, R⁹, R¹⁰, and X are as defined therein.

Particularly preferred compounds of formula (XII) are

(3S,5R,6S)-3-[2-cyclopropoxy-5-(trifluoromethoxy)phenyl]-6-phenyl-1-oxa-7-aza-spiro[4.5]decane;

(3R,5R,6S)-3-[2-cyclopropoxy-5-(trifluoromethoxy)phenyl]-6-phenyl-1-oxa-7-aza-spiro[4.5]decane; or a pharmaceutically acceptable salt thereof.

Another class of tachykinin receptor antagonists of use in the present invention is that described in PCT International Patent Publication No. WO 95/06645 as compounds of formula (XIII):

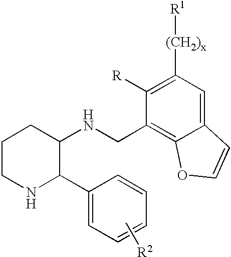

wherein

R represents a hydrogen atom or a C₁₋₄ alkoxy group;

R¹ is selected from phenyl, optionally substituted by a group —(CH₂)ₙCONR³R⁴ or S(O)ₘR³; or a 5-or 6-membered aromatic heterocycle containing 1, 2, 3 or 4 heteroatoms selected from oxygen, nitrogen, or sulphur, optionally substituted by a C₁₋₄ alkyl, trifluoromethyl or cyano group or a group —(CH₂)ₙCONR³R⁴;

R² represents a hydrogen or halogen atom;

R³ and R⁴ independently represent hydrogen or C₁₋₄ alkyl;

n represents zero, 1 or 2;

m represents zero, 1 or 2;

z represents zero or 1;

and pharmaceutically acceptable salts and solvates thereof.

A particularly preferred compound of formula (XIII) is [5-(5-methyl-tetrazol-1-yl)-benzofuran-7-ylmethyl]-(2S-phenyl-piperidin-3S-yl)-amine; or a pharmaceutically acceptable salt thereof.

Another class of tachykinin receptor antagonists of use in the present invention is that described in PCT International Patent Publication No. WO 95/14017, i.e. compounds of formula (XIV)

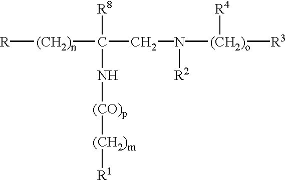

or a pharmaceutically acceptable salt thereof, wherein

m is zero, 1, 2 or 3;

n is zero or 1;

o is zero, 1 or 2;

p is zero or 1;

R is phenyl, 2-or 3-indolyl, 2-or 3-indolinyl, benzothienyl, benzofuranyl, or naphthyl;

which R groups may be substituted with one or two halo, C₁₋₃alkoxy, trifluoromethyl, C₁₋₄alkyl, phenyl-C₁₋₃alkoxy, or C₁₋₄alkanoyl groups;

R¹ is trityl, phenyl, diphenylmethyl, phenoxy, phenylthio, piperazinyl, piperidinyl, pyrrolidinyl, morpholinyl, indolinyl, indolyl, benzothienyl, hexamethyleneiminyl, benzofuranyl, tetrahydropyridinyl, quinolinyl, isoquinolinyl, reduced quinolinyl, reduced isoquinolinyl, phenyl-(C₁₋₄alkyl)-, phenyl-(C₁₋₄alkoxy)-, quinolinyl-(C₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-(C₃alkyl)-, C₁₋₄alkyl, or —NH—CH₂—R⁵;

any one of which R¹ groups may be substituted with halo, C₁₋₄alkyl, C₁₋₄alkoxy, trifluoromethyl, amino, C₁₋₄alkylamino, di(C₁₋₄alkyl)amino, or C₂₋₄alkanoylamino;

or any one of which R¹ groups may be substituted with phenyl, piperazinyl, C₃₋₈cycloalkyl, benzyl, C₁₋₄alkyl, piperidinyl, pyridinyl, pyrimidinyl, C₂₋₆alkanoylamino, pyrrolidinyl, C₂₋₆alkanoyl, or C₁₋₄alkoxycarbonyl;

any one of which groups may be substituted with halo, C₁₋₄alkyl, C₁₋₄alkoxy, trifluoromethyl, amino, C₁₋₄alkylamino, di(C₁₋₄alkyl)amino, or C₂₋₄alkanoylamino;

or R¹ is amino, a leaving group, hydrogen, C₁₋₄alkylamino, or di(C₁₋₄alkyl)amino;

R⁵ is pyridyl, anilino-(C₁₋₃alkyl)-, or anilinocarbonyl;

R² is hydrogen, C₁₋₄alkyl, C₁₋₄alkylsulfonyl, carboxy-(C₁₋₃alkyl)-, C₁₋₃alkoxycarbonyl-(C₁₋₃alkyl)-, or —CO—R⁶;

R⁶ is hydrogen, C₁₋₄alkyl, C₁₋₃haloalkyl, phenyl, C₁₋₃alkoxy, C₁₋₃hydroxyalkyl, amino, C₁₋₄alkylamino, di(C₁₋₄alkyl)amino, or —(CH₂)_{q}—R⁷;

q is zero to 3;

R⁷ is carboxy, C₁₋₄alkoxycarbonyl, C₁₋₄alkylcarbonyloxy, amino, C₁₋₄alkylamino, di(C₁₋₄alkyl)amino, C₁₋₆alkoxycarbonylamino, or phenoxy, phenylthio, piperazinyl, piperidinyl, pyrrolidinyl, morpholinyl, indolinyl, indolyl, benzothienyl, benzofuranyl, quinolinyl, phenyl-(C₁₋₄alkyl)-, quinolinyl-(C₁₋₄alkyl)-, isoquinolinyl-(C₁₋₄alkyl)-, reduced quinolinyl-(C₁₋₄alkyl)-, reduced isoquinolinyl-(C₁₋₄alkyl)-, benzoyl-C₁₋₃alkyl;

any one of which aryl or heterocyclic R⁷ groups may be substituted with halo, trifluoromethyl, C₁₋₄alkoxy, C₁₋₄alkyl, amino, C₁₋₄alkylamino, di(C₁₋₄alkyl)amino, or C₂₋₄alkanoylamino;

or any one of which R⁷ groups may be substituted with phenyl, piperazinyl, C₃₋₈cycloalkyl, benzyl, piperidinyl, pyridinyl, pyrimidinyl, pyrrolidinyl, C₂₋₆alkanoyl, or C₁₋₄alkoxycarbonyl;

any of which groups may be substituted with halo, trifluoromethyl, amino, C₁₋₄alkoxy, C₁₋₄alkyl, C₁₋₄alkylamino, di(C₁₋₄alkyl)amino, or C₂₋₄alkanoylamino;

R⁸ is hydrogen or C₁₋₆alkyl;

R³ is phenyl, phenyl-(C₁₋₆alkyl)-, C₃₋₈cycloalkyl, C₅₋₈cycloalkenyl, C₁₋₈alkyl, naphthyl, C₂₋₈alkenyl, or hydrogen;

any one or which groups except hydrogen may be substituted with one or two halo, C₁₋₃alkoxy, C₁₋₃alkylthio, nitro, trifluoromethyl, or C₁₋₃alkyl groups; and

R⁴ is hydrogen or C₁₋₃alkyl;

with the proviso that if R¹ is hydrogen or halo, R³ is phenyl, phenyl-(C₁₋₆alkyl)-, C₃₋₈cycloalkyl, C₅₋₈cycloalkenyl, or naphthyl.

A particularly preferred compound of formula (XIV) is [N-(2-methoxybenzyl)acetylamino]-3-(1-indol-3-yl)-2-[N-(2-(4-piperidin-1-yl)piperidin-1-yl)acetylamino]propane; or a pharmaceutically acceptable salt thereof.

The preferred compounds of formulae (I), (II), (III) and (IV) will have the 2- and 3-substituents on the morpholine ring in the cis arrangement, the preferred stereochemistry being as shown in the following general formula:

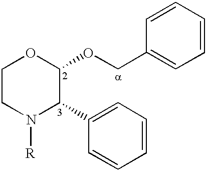

Where the benzyloxy moiety is α-substituted, the preferred stereochemistry of the α-carbon is either (R) when the substituent is an alkyl (e.g. methyl) group or (S) when the substituent is a hydroxyalkyl (e.g. hydroxymethyl) group.

Preferred NK-1 receptor antagonis ts for use in the present invention as orally active, long acting, CNS-penetrant NK-1 receptor antagonists are selected from the classes of compounds described in European Patent Specification No. 0 577 394, and International Patent Specification Nos. 95/08549, 95/18124, 95/23798, 96/05181 and 97/49710.

Particularly preferred NK-1 receptor antagonists of use in the present invention include:

(±)-(2R3R, 2S3S)-N-{[2-cyclopropoxy-5-(trifluoromethoxy)-phenyl]methyl)-2-phenyl]piperidin-3-amine;

2-(S)-(3,5-bis(trifluoromethyl)benzyloxy)-3(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;

2-(S)-(3,5-bis(trifluoromethyl)benzyloxy)-4-(3-(5-oxo-1H,4H-1,2,4-triazolo)methyl)-3-(S)-phenyl-morpholine;

2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine;

2-(R)-(1-(R)-(3,5-bis(trifluoromethyl)phenyl)ethoxy)-4-(5-(N,N-dimethylamino)methyl-1,2,3-triazol-4-yl)methyl-3-(S)-(4-fluorophenyl)morpholine;

(3S,5R,6S)-3-[2-cyclopropoxy-5-(trifluoromethoxy)phenyl]-6-phenyl-1-oxa-7-aza-spiro[4.5]decane;

(3R,5R,6S)-3-[2-cyclopropoxy-5-(trifluoromethoxy)phenyl]-6-phenyl-1-oxa-7-aza-spiro[4.5]decane;

2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;

2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;

2-(R)-(1-(R)-(3,5-bis(trifluoromethyl)phenyl)ethoxy)-3-(S)-(4-25-fluorophenyl)-4-(3-(2-monophosphoryl-5-oxo-1H-1,2,4-triazolo)methyl)morpholine;

2-(R)-(1-(R)-3,5-bis(trifluoromethyl)phenyl)ethoxy)-3-(S)-(4-fluorophenyl)-4-(3-(5-oxy)hosphoryl-1H-1,2,4-triazolo)methyl)morpholine;

2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;

2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;

or a pharmaceutically acceptable salt thereof

Full descriptions of the preparation of the tachykinin receptor antagonists which may be employed in the present invention may be found in the references cited herein.

Unless otherwise defined herein, suitable alkyl groups include straight-chained and branched alkyl groups containing from 1 to 6 carbon atoms. Typical examples include methyl and ethyl groups, and straight-chained or branched propyl and butyl groups. Particular alkyl groups are methyl, ethyl, n-propyl, isopropyl, n-butyl, sec-butyl and tert-butyl.

Unless otherwise defined herein, suitable alkenyl groups include straight-chained and branched alkenyl groups containing from 2 to 6 carbon atoms. Typical examples include vinyl and allyl groups.

Unless otherwise defined herein, suitable alkynyl groups include straight-chained and branched alkynyl groups containing from 2 to 6 carbon atoms. Typical examples include ethynyl and propargyl groups.

Unless otherwise defined herein, suitable cycloalkyl groups include groups containing from 3 to 7 carbon atoms. Particular cycloalkyl groups are cyclopropyl and cyclohexyl.

Unless otherwise defined herein, suitable aryl groups include phenyl and naphthyl groups. A particular aryl-C₁₋₆alkyl, e.g. phenyl-C₁₋₆alkyl, group is benzyl.

Unless otherwise defined herein, suitable heteroaryl groups include pyridyl, quinolyl, isoquinolyl, pyridazinyl, pyrimidinyl, pyrazinyl, pyranyl, furyl, benzofuryl, thienyl, benzthienyl, imidazolyl, oxadiazolyl and thiadiazolyl groups.

The term “halogen” as used herein includes fluorine, chlorine, bromine and iodine. The compounds of use in this invention may have one or more asymmetric centres and can therefore exist as enantiomers and possibly as diastereoisomers. It is to be understood that the present invention relates to the use of all such isomers and mixtures thereof.

Suitable pharmaceutically acceptable salts of the NK-1 receptor antagonists of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulphuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof

The above compounds are only illustrative of the neurokinin-1 antagonists which are currently under investigation. As this listing of compounds is not meant to be comprehensive, the methods of the present invention may employ any neurokinin-1 receptor antagonist, in particular a neurokinin-1 receptor antagonist which is orally active, long acting and CNS-penetrant. Accordingly, the present invention is not strictly limited to any particular structural class of compound.

Certain of the above defined terms may occur more than once in the above formula and upon such occurrence each term shall be defined independently of the other. Similarly, the use of a particular variable within a noted structural formula is intended to be independent of the use of such variable within a different structural formula.

Full descriptions of the preparation of the tachykinin receptor antagonists which are employed in the present invention may be found in the references cited herein.

The present invention accordingly provides the use of a NK-1 receptor antagonist selected from the compounds of formulae (I), (II), (III), (IV), (V), (VI), (VII), (VIII), (IX), (X), (XI), (XII), (XIII) and (XIIV) for the manufacture of a medicament for alleviating or managing symptoms associated with premenstrual syndrome, in particular treating or preventing disturbances of mood associated with premenstrual syndrome, in a woman.

The present invention also provides a method for alleviating or managing symptoms associated with premenstrual syndrome, in particular treating or preventing disturbances of mood associated with premenstrual syndrome, in a woman, which method comprises administration to a patient in need of such treatment an effective amount of a NK-1 receptor antagonist selected from the compounds of formulae (I), (II), (III), (IV), (V), (VI), (VII), (VIII), (IX), (X), (XI), (XII), (XIII) and (XIIV).

In a further aspect of the present invention, there is provided a pharmaceutical composition for alleviating or managing symptoms associated with premenstrual syndrome, in particular treating or preventing disturbances of mood associated with premenstrual syndrome, in a woman comprising aL NK-1 receptor antagonist selected from the compounds of formulae (I), (II), (III), (IV), (V), (VI), (VII), (VIII), (IX), (X), (XI), (XII), (XIII) and (XIIV), together with at least one pharmaceutically acceptable carrier or excilpient.

The identification of a compound as a tachykinin receptor antagonist, in particular, a neurokinin-1 receptor antagonist, and thus able to have utility in the present invention may be readily determined without undue experimentation by methodology well known in the art.

A tachykinin receptor antagonist may be administered alone or in combination by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Preferably the compositions according to the present invention are in unit dosage forms such as tablets, pills, capsules, powders, granules, solutions or suspensions, or suppositories, for oral, parenteral or rectal administration, by inhalation or insufflation or administration by trans-dermal patches or by buccal cavity absorption wafers.

For preparing solid compositions such as, tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Preferred compositions for administration by injection include those comprising a NK-1 receptor antagonist as the active ingredient, in association with a surface-active agent (or wetting agent or surfactant) or in the form of an emulsion (as a water-in-oil or oil-in-water emulsion).

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and preferably between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion will preferably comprise fat droplets between 0.1 and 1.0 μm, particularly 0.1 and 0.5 μm, and have a pH in the range of 5.5 to 8.0.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulised by use of inert gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

Compositions of the present invention may also be presented for administration in the form of trans-dermal patches using conventional technology. The compositions may also be administered via the buccal cavity using, for example, absorption wafers.

Compositions in the form of tablets, pills, capsules or wafers for oral administration are particularly preferred.

It will be known to those skilled in the art that there are numerous compounds now being used for alleviating or managing symptoms associated with premenstrual syndrome, in particular treating or preventing disturbances of mood associated with premenstrual syndrome, in a woman. Combinations of these therapeutic agents some of which have also been mentioned herein with a tachykinin receptor antagonist will bring additional, complementary, and often synergistic properties to enhance the desirable properties of these various therapeutic agents. In these combinations, the tachykinin receptor antagonist and the therapeutic agents may be independently present in dose ranges from one one-hundredth to one times the dose levels which are effective when these compounds are used singly.

The tachykinin receptor antagonist may be administered in combination with sedatives, hypnotics, anxiolytics, antipsychotics, antianxiety agents, estrogens, estrogen receptor modulators, estrogen agonists, minor tranquilizers, benzodiazepines, barbituates, serotonin agonists, selective serotonin reuptake inhibitors, 5HT-2 antagonists, non-steroidal anti-inflammatory drugs, oral contraceptives, progesterone, progestin, monoamine oxidase inhibitors, carbohydrate mixtures and the like, or the tachykinin receptor antagonist may be administered in conjunction with the use of physical methods such as electrical stimulation.

For example, for alleviating or managing symptoms associated with premenstrual syndrome, in particular treating or preventing disturbances of mood associated with premenstrual syndrome, in a woman a tachykinin receptor antagonist may be given in combination with such compounds as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clometherone, clomipramine, cloperidone, clorazepate, clorethate, clozapine, cyprazepam, delmadinone, desipramine, dexclamol, diazepam, dichloralphenazone, divalproex, diphenhydramine, doxepin, droloxifene, estazolam, estradiol, estrogen, ethchlorvynol, etomidate, fenobam, flunitrazepam, flurazepam, fluvoxamine, fluoxetine, fosazepam, glutethimide, halazepam, hydroxyzine, idoxifene, imipramine, lithium, leuprolide, lorazepam, lormetazepam, maprotiline, mecloqualone, melatonin, mephobarbital, meprobamate, methaqualone, midaflur, midazolam, nafoxidine, nefazodone, nitromifene, nisobamate, nitrazepam, nortriptyline, ormeloxifene, oxazepam, paraldehyde, paroxetine, pentobarbital, perlapine, perphenazine, phenelzine, phenobarbital, prazepam, progesterone, promethazine, propofol, protriptyline, quazepam, raloxifene, reclazepam, roletamide, secobarbital, sertraline, suproclone, tamoxifene, temazepam, thioridazine, toremifene, tracazolate, tranylcypromaine, trazodone, trioxifene, triazolam, trepipam, tricetamide, triclofos, trifluoperazine, trimetozine, trimipramine, uldazepam, valproate, venlafaxine, zaleplon, zolazepam, zolpidem, and salts thereof, and combinations thereof, and the like, as well as admixtures and combinations thereof.

Typically, the individual daily dosages for these combinations may range from about one-fifth of the minimally recommended clinical dosages to the maximum recommended levels for the entities when they are given singly. In the case of fluoxetine the recommended clinical dose is generally from approximately 5 mg/day to approximately 120 mg/day. For d-fenfluramine or d,l-fenfluramine, the recommended clinical dose is generally from approximately 7 mg/day to approximately 60 mg/day is administered. In the case of fluvoxamine, the recommended clinical dose is generally from about 25 mg/day given once daily, to about 200 mg/day given in two 100 mg doses, is administered. In the case of sertraline, the recommended clinical dose is generally from about 50 mg/day to approximately 150 mg/day is administered.

Preferred agents for use in combination with a tachykinin receptor antagonist, in particular a neurokinin-1 receptor antagonist, include fluoxetine, fluvoxamine and sertraline.

To illustrate these combinations, a tachykinin receptor antagonist effective clinically at a given daily dose range may be effectively combined, at levels which are equal or less than the daily dose range, with the aforementioned compounds. It will be readily apparent to one skilled in the art that the tachykinin receptor antagonist may be employed with other agents for alleviating or managing symptoms associated with premenstrual syndrome, in particular treating or preventing disturbances of mood associated with premenstrual syndrome, in a woman.

Naturally, these dose ranges may be adjusted on a unit basis as necessary to permit divided daily dosage and, as noted above, the dose will vary depending on the nature and severity of the disease, weight of patient, special diets and other factors. These combinations may be formulated into pharmaceutical compositions as known in the art and as discussed herein.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease or disorder, the patient's weight, special diets then being followed by a patient, concurrent medication, the intrinsic tachykinin receptor antagonist activity of the compound, the bioavailability upon oral administration of the compound and other factors which those skilled in the art will recognize.

In the treatment of a condition in accordance with the present invention, an appropriate dosage level will generally be about 0.01 mg to 50 mg per kg patient body weight per day which may be administered in single or multiple doses. Preferably, the dosage level will be about 0.1 mg to about 25 mg/kg per day; more preferably about 0.5 mg to about 10 mg/kg per day. For example, for alleviating or managing symptoms associated with premenstrual syndrome, in particular treating or preventing disturbances of mood associated with premenstrual syndrome, in a woman, a suitable dosage level is about 0.1 mg to 25 mg/kg per day, preferably about 0.5 mg to 10 mg/kg per day, and especially about 1 mg to 5 mg/kg per day. In larger mammals, for example humans, a minimum oral dosage level is about 1 mg/day, preferably about 5 mg per day and especially about 10 mg per day, and a maximum oral dosage level is about 1500 mg per day, preferably about 1000 mg per day and especially about 500 mg per day. A compound may be administered on a regimen of several times per day, for example 1 to 4 times per day, preferably once or twice per day. When using an injectable formulation, a suitable dosage level is about 0.1 mg to 10 mg/kg per day, preferably about 0.5 mg to 5 mg/kg per day, and especially about 1 μg to 1 mg/kg per day. In larger mammals, for example humans, a typical indicated dose is about 100 mg to 100 mg i.v. A compound may be administered on a regimen of several times per day, for example 1 to 4 times per day, preferably once or twice per day, and more preferably once a day.

Pharmaceutical compositions of the present invention may be provided in a solid dosage formulation preferably comprising about 100 μg to 500 mg active ingredient, more preferably comprising about 100 μg to 250 mg active ingredient. The pharmaceutical composition is preferably provided in a solid dosage formulation comprising about 100 μg, 1 mg, 5 mg, 10 mg, 25 mg, 50 mg, 100 mg, 200 mg or 300 mg active ingredient. A minimum dosage level for the NK-1 receptor antagonist is generally about 5 mg per day, preferably about 10 mg per day and especially about 20 mg per day. A maximum dosage level for the NK-1 receptor antagonist is generally about 1500 mg per day, preferably about 1000 mg per day and especially about 500 mg per day.

It will be appreciated that the amount of the NK-1 receptor antagonist required for use in alleviating or managing symptoms associated with premenstrual syndrome, in particular treating or preventing disturbances of mood associated with premenstrual syndrome, in a woman will vary not only with the particular compounds or compositions selected but also with the route of administration, the nature of the condition being treated, and the age and condition of the patient, and will ultimately be at the discretion of the patient's physician or pharmacist.

The length of time during which a tachykinin receptor antagonist will be given varies on an individual basis, but will generally begin 1 to 14 days prior to menstruation and may continue for several days (e.g., 3 days) after onset of menstruation. Because symptoms associated with premenstrual syndrome may disappear after the onset of menses (except in perimenopausal women), however, it is preferred that the tachykinin receptor antagonist be administered to the woman prior to the onset of her menstrual period.

The compounds of formulae (I), (II), (III), (IV), (V), (VI), (VII), (VIII), (IX), (X), (XI), (XII) and (XIII) may be prepared by the methods described in EP-A-0 577 394 (or WO 95/16679), WO 95/18124, WO 95/23798, WO 96/05181, EP-A-0 436 334, WO 93/21155, EP-A-0 591 040, EP-A-0 532 456, EP-A-0 443 132, WO 92/17449, WO 95/08549, WO 95/06645 and WO 95/14017, respectively.

Particularly preferred NK-1 receptor antagonists of the formulae (I), (II), (III), (IV), (V), (VI), (VII), (VIII), (IX), (X), (XI), (XII), (XIII) and (XIIV) for use in the present invention are compounds which are potent NK-1 receptor antagonists, i.e. compounds with an NK-1 receptor affinity (IC₅₀) of less than 10 nM.

A particularly preferred class of NK-1 receptor antagonist of use in the present invention are those compounds which are orally active, long acting and CNS-penetrant. Such compounds may be identified using the pharmacological assays described hereinafter. The use of this sub-class of NK-1 antagonists for alleviating or managing symptoms associated with premenstrual syndrome, in particular treating or preventing disturbances of mood associated with premenstrual syndrome, in a woman represents a further aspect of the present invention.

Thus, the present invention provides the use of a CNS penetrant NK-1 receptor antagonist in an oral, once-a-day medicament for alleviating or managing symptoms associated with premenstrual syndrome, in particular treating or preventing disturbances of mood associated with premenstrual syndrome, in a woman. The compounds of this class advantageously exhibit a rapid onset of action and a reduced side-effect profile when compared against conventional treatments for alleviating or managing symptoms associated with premenstrual syndrome, in particular treating or preventing disturbances of mood associated with premenstrual syndrome, in a woman.

In particular, the present invention provides a means for the identification of NK-1 receptor antagonists which would be especially effective in an oral once-a-day medicament for alleviating or managing symptoms associated with premenstrual syndrome, in particular treating or preventing disturbances of mood associated with premenstrual syndrome, in a woman.

The exceptional pharmacology of the class of NK-1 receptor antagonists of use in the present invention enables alleviating or managing symptoms associated with premenstrual syndrome, in particular treating or preventing disturbances of mood associated with premenstrual syndrome, in a woman, without the need for concomitant therapy and in particular, without the need for concomitant use of a serotonin agonist or an SSRI.

Furthermore, the exceptional pharmacology of the class of NK-1 receptor antagonists of use in the present invention results in a rapid onset of action.

The present invention accordingly provides the use of an orally active, long acting, CNS-penetrant NK-1 receptor antagonist (as hereinafter defined) for the manufacture of a medicament adapted for oral administration for alleviating or managing symptoms associated with premenstrual syndrome, in particular treating or preventing disturbances of mood associated with premenstrual syndrome, in a woman.

The present invention also provides a method for the for alleviating or managing symptoms associated with premenstrual syndrome, in particular treating or preventing disturbances of mood associated with premenstrual syndrome, in a woman, which method comprises the oral administration to a patient in need of such treatment of an effective amount of an orally active, long acting, CNS-penetrant NK-1 receptor antagonist (as defined herein).

The present invention is useful for alleviating or managing adverse symptoms in a woman prior to menstruation, such as disturbances of appetite and/or disturbances of mood. A preferred embodiment of the invention, is directed to a method for alleviating or managing the symptoms in a patient suffering from premenstrual or late luteal phase syndrome rather than the more severe and debilitating disorder characterized as premenstrual dysphoric disorder. Accordingly, it is preferred that the patient be suffering from other than premenstrual dysphoric disorder.

In a further aspect of the present invention, there is provided an oral pharmaceutical composition for alleviating or managing symptoms associated with premenstrual syndrome, in particular treating or preventing disturbances of mood associated with premenstrual syndrome, in a woman which comprises an orally active, long acting, CNS-penetrant NK-1 receptor antagonist (as hereinafter defined), together with a pharmaceutically acceptable carrier or excipient.

It will be appreciated to those skilled in the art that reference herein to treatment extends to prophylaxis (prevention) as well as the treatment of the noted diseases/disorders and symptoms.

The following examples are provided for the purpose of further illustration only and are not intended to be limitations on the disclosed invention.

Particularly preferred NK-1 receptor antagonists of use in the present invention are compounds which are potent NK-1 receptor antagonists, i.e. compounds with an NK-1 receptor affinity (IC₅₀) of less than 10 nM, favourably less than 2nM and preferably less than 1 nM.

The class of orally active, long acting, CNS-penetrant NK-1 receptor antagonists of use in the present invention is identified using a combination of the following assays:

### EXAMPLE 1

NK-1 Receptor Binding Assay

NK-1 receptor binding assays are performed in intact Chinese hamster ovary (CHO) cells expressing the human NK-1 receptor using a modification of the assay conditions described by Cascieri et al, J. Pharmacol. Exp. Ther., 1992, 42, 458. Typically, the receptor is expressed at a level of 3×10⁵ receptors per cell. Cells are grown in monolayer culture, detached from the plate with enzyme-free dissociation solution (Speciality Media Inc.), and washed prior to use in the assay. ¹²⁵I-Tyr⁸-substance substance P (0.1 nM, 200Ci/mmol; New England Nuclear) is incubated in the presence or absence of test compounds (dissolved in 5 μl dimethylsulphoxide, DMSO) with 5×10⁴ CHO cells. Ligand binding is performed in 0.25 ml of 50 mM Tris-HCl, pH7.5, containing 5 mM MnCl₂, 150 mM NaCl, 0.02% bovine serum albumin (Sigma), 50 μg/ml chymostatin (Peninsula), 0.1 nM phenylmethylsulphonyl fluoride, 2 μg/ml pepstatin, 2μg/ml leupeptin and 2.8 μg/ml furoyl saccharine. The incubation proceeds at room temperature until equilibrium is achieved (>40 minutes) and the receptor-ligand complex is harvested by filtration over GF/C filters pre-soaked in 0.1% polyethylenimine using a Tomtek 96-well harvester. Non-specific binding is determined using excess substance P (1 μM) and represents <10% of total binding.

### EXAMPLE 2

Gerbil Foot-Tapping Assay

CNS penetrant NK-1 receptor antagonists for use in the present invention can be identified by their ability to inhibit foot tapping in gerbils induced by anxiogenic agents (such as pentagastrin) or central infusion of NK-1 receptor agonists such as GR73632, or caused by aversive stimulation such as foot shock or single housing, based on the method of Rupniak & Williams, Eur. J. Pharmacol., 1994, 265, 179.

Male or female Mongolian gerbils (35-70 g) are anaesthetised by inhalation of an isoflurane/oxygen mixture to permit exposure of the jugular vein in order to permit administration of test compounds or vehicle in an injection volume of 5 ml/kg i.v. Alternatively, test compounds may be administered orally or by subcutaneous or intraperitoneal routes. The wound is closed and a second skin incision is made in the midline of the scalp to expose the skull. An anxiogenic agent (e.g. pentagastrin) or a selective NK-1 receptor agonist (e.g. GR73632 (d Ala[L-Pro⁹,Me-Leu¹⁰]-substance P-(7-11))) is infused directly into the cerebral ventricles (e.g. 3 pmol in 5 μl i.c.v. depending upon the agent) by vertical insertion of a cuffed 27 gauge needle to a depth of 4.5 mm below bregma. The scalp incision is closed and the animal allowed to recover from anaesthesia in a clear perspex observation box (25cm×20cm×20cm). The duration and/or intensity of hind foot tapping is then recorded continuously for 5 minutes. Alternatively, the ability of test compounds to inhibit foot tapping evoked by aversive stimulation, such as foot shock or single housing, may be studied using a similar method of quantification.

### EXAMPLE 3

Ferret Emesis Assay

Individually housed male ferrets (1.0-2.5 kg) are dosed orally by gavage with test compound. Ten minutes later they are fed with approximately 100 g of tinned cat food. At 60 minutes following oral dosing, cisplatin (10 mg(kg) is given i.v. via a jugular vein catheter inserted under a brief period of halothane anaesthesia. The catheter is then removed, the jugular vein ligated and the skin incision closed. The ferrets recover rapidly from the anaesthetic and are mobile within 10-20 minutes. The animals are observed continuously during recovery from the anaesthetic and for 4 hours following the cisplatin injection, after which time the animals are killed humanely. The numbers of retches and vomits occurring during the 4 hours after cisplatin administration are recorded by trained observers.

### EXAMPLE 4

Separation-Induced Vocalisation Assay

Male and female guinea-pigs pups are housed in family groups with their mothers and littermates throughout the study. Experiments are commenced after weaning when the pups are 2 weeks old. Before entering an experiment, the pups are screened to ensure that a vigorous vocalisation response is reproducibly elicited following maternal separation. The pups are placed individually in an observation cage (55 cm×39 cm×19 cm) in a room physically isolated from the home cage for 15 minutes and the duration of vocalisation during this baseline period is recorded. Only animals which vocalise for longer than 5 minutes are employed for drug challenge studies (approximately 50% of available pups may fail to reach this criterion). On test days each pup receives an oral dose or a s.c. or i.p. injection of test compound or vehicle and is then immediately returned to the home cage with its mother and siblings for 30 minutes to 60 minutes (or for up to 4 hours following an oral dose, dependant upon the oral pharmacokinetics of the test compound) before social isolation for 15 minutes as described above. The duration of vocalisation on drug treatment days is expressed as a percentage of the pre-treatment baseline value for each animal. The same subjects are retested once weekly for up to 6 weeks. Between 6 and 8 animals receive each test compound at each dose tested.

A suitable selection cascade for NK₁ antagonists of use according to the present invention is as follows:

(i) Determine affinity for human NK₁ receptor in radioligand binding studies (Assay 1); select compounds with IC₅₀≦10 nM, preferably IC₅₀≦2 nM, especially IC₅₀≦1 nM.

(ii) Determine ability of compounds to penetrate CNS by their ability to inhibit foot tapping in gerbils induced by central injection of an NK₁ agonist (Assay 2); select compounds that inhibit foot tapping with ID₅₀≦3 mg/kg i.v., and preferably ID₅₀≦1 mg/kg i.v. when administered immediately prior to central NK₁ agonist challenge, or ID₅₀≦30 mg/kg p.o., and preferably ID₅₀≦10 mg/kg p.o. 1 hour prior to challenge.

(iii) Determine central duration of action of compounds in gerbil foot tapping assay following intravenous administration 24 hours prior to central NK₁ agonist challenge; select compounds showing ≦25-fold loss of potency compared with ID₅₀ determined in step (ii) above with the proviso that ID₅₀≦10 mg/kg i.v., and preferably ≦5 mg/kg i.v. after 24 hour pre-treatment.

(iv) Determine oral bioavailability of compounds by pharmacokinetic analysis, activity in gerbil foot tapping assay following oral administration and/or by ability to inhibit cisplatin-induced emesis in ferrets (Assay 3); select compounds with ID₉₀≦3 mg/kg p.o., and preferably ID₉₀≦1 mg/kg p.o.

Particularly preferred compounds of use in the present invention are identified using steps (i) to (iv) followed by step (v):

(v) Determine activity of compounds in assays sensitive to conventional antidepressant drugs (inhibition of pharmacologically evoked foot tapping in gerbils and/or inhibition of distress vocalisations in guinea-pig pups (Assay 4)). Select compounds with ID₅₀≦20 mg/kg, and preferably ID₅₀≦10 mg/kg.

Yet further preferred compounds of use in the present invention may be selected from those compounds which satisfy the NK-1 receptor binding criteria of step (i) which, in addition, have ≦5-fold shift in affinity when incubated in the presence of human serum albumin (HSA) to show non-specific protein binding.

One example of a NK-1 receptor antagonist of use in the present invention is the compound 2-(R)-(1-(R)-(3,5-bis(trifluoromethyl)-phenyl)-ethoxy)-3-(S)-(4-fluorophenyl)-4-(3-(5-oxo-1H,4H-1,2,4-triazolo)methyl)-morpholine, the preparation of which is described in PCT Patent Publication No. WO 95/16679. In the aforementioned assays, this compound has the following activity:

| | |
|---|---|
| human NK-1 receptor binding: | IC₅₀ = 0.1 nM |
| gerbil foot-tapping (5 mins.): | IC₅₀ = 0.36 mg/kg i.v. |
| gerbil foot-tapping (24 hrs.): | IC₅₀ = 0.33 mg/kg i.v. |
| ferret emesis: | ID₉₀ = <3 mg/kg p.o. |
| guinea-pig vocalisation | ID₅₀ = 0.73 mg/kg p.o. |
| (4 hr. pre-treatment): | |

### EXAMPLE 5

Double-Blind, Placebo-Controlled Study to Determine the Effect of a Substance P Antagonist on Mood and Appetite Disturbances Associated with Premenstrual Syndrome in Women.

Approximately twenty women receive either the substance P receptor antagonist 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl-morpholine (30 mg/day) or a placebo starting approximately two weeks prior to their expected menstrual period. Each subject participates in 6 randomized test periods; in 3 of the test periods, each is given the substance P antagonist and in the other 3 test periods, is given a placebo. Mood is assessed 1-3 days before the onset of menses, using the Hamilton Depression Scale and the PMS/LLPD Symptom Rating Scale, for mood and appetite symptoms. Hamilton, N., Journal of Neurosurgery and Psychiatry, 23:56-62 (1960); Steiner, M. et al., Acta Psychiatrica Scandinavia, 62:177-190 (1980). Food intake is measured through the use of self-reports (when subjects were out-patients), and directly (while subjects were inpatients), during one drug and one placebo period; subjects also are weighed. The results between the test periods are compared and the results for each subject are evaluated. The results of the foregoing study would indicate that the administration of a substance P antagonist should have a positive effect with respect to placebo in decreasing depression and other negative mood states (such as tension, anger, confusion, and irritability) following drug treatment.

The following examples illustrate pharmaceutical compositions according to the invention.

### EXAMPLE 6

Tablet formulation containing 50-300 mg of NK-1 antagonist

| | Amount mg | | |
|---|---|---|---|
| NK-1 antagonist | 50.0 | 100.0 | 300.0 |
| Microcrystalline cellulose | 80.0 | 80.0 | 80.0 |
| Modified food corn starch | 80.0 | 80.0 | 80.0 |
| Lactose | 189.5 | 139.5 | 439.5 |
| Magnesium Stearate | 0.5 | 0.5 | 0.5 |

The active ingredient, cellulose, lactose and a portion of the corn starch are mixed and granulated with 10% corn starch paste. The resulting granulation is sieved, dried and blended with the remainder of the corn starch and the magnesium stearate. The resulting granulation is then compressed into tablets containing 50 mg, 100 mg and 300 mg of the NK-1 receptor antagonist per tablet.

### EXAMPLE 7

Parenteral injection formulation

| | Amount | |
|---|---|---|
| Active Ingredient | 10 to 300 | mg |
| Citric Acid Monohydrate | 0.75 | mg |
| Sodium Phosphate | 4.5 | mg |
| Sodium Chloride | 9 | mg |
| Water for injection | to 10 | ml |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for alleviating or managing symptoms associated with premenstrual syndrome which comprises administering to a woman in need thereof an effective amount of a tachykinin receptor antagonist.

2. The method of claim 1 wherein the tachykinin receptor antagonist is a neurokinin-1 receptor antagonist.

3. The method of claim 2 wherein the neurokinin-1 receptor antagonist is a non-peptidal neurokinin-1 receptor antagonist.

4. The method of claim 3 wherein the neurokinin-1 receptor antagonist is a CNS-penetrating neurokinin-1 receptor antagonist.

5. The method of claim 4 wherein the neurokinin-1 receptor antagonist is an orally active neurokinin-1 receptor antagonist.

6. The method of claim 5 wherein the neurokinin-1 receptor antagonist possesses a long duration of action.

7. A method for the treatment or prevention of disturbances of appetite, disturbances of mood, or both, associated with premenstrual syndrome which comprises administering to a woman in need thereof an effective amount of a tachykinin receptor antagonist.

8. The method of claim 7 wherein the tachykinin receptor antagonist is a neurokinin-1 receptor antagonist.

9. The method of claim 8 wherein the neurokinin-1 receptor antagonist is a non-peptidal neurokinin-1 receptor antagonist.

10. The method of claim 9 wherein the neurokinin-1 receptor antagonist is a CNS-penetrating neurokinin-1 receptor antagonist.

11. The method of claim 10 wherein the neurokinin-1 receptor antagonist is an orally active neurokinin-1 receptor antagonist.

12. The method of claim 11 wherein the neurokinin-1 receptor antagonist possesses a long duration of action.
